(19) United States
(12) Patent Application Publication
(10) Pub. No.: US 2026/0159488 A1
(43) Pub. Date: Jun. 11, 2026

(54) SYNTHESIS OF QUINOXALINES FROM O-PHENYLENEDIAMINES AND SULFOXONIUM YLIDES

(71) Applicant: UNIVERSITY OF TABUK, Tabuk (SA)

(72) Inventors: Humaira PARVEEN, Tabuk (SA); Sayeed MUKHTAR, Tabuk (SA); Mona Obead Salman ALBALAWI, Tabuk (SA); Mohmmad Younus WANI, Tabuk (SA)

(73) Assignee: UNIVERSITY OF TABUK, Tabuk (SA)

(21) Appl. No.: 19/033,327

(22) Filed: Jan. 21, 2025

(30) Foreign Application Priority Data
Dec. 9, 2024 (SA) 1020246984

(51) Int. Cl. C07D 241/42; C07D 239/28; C07D 405/04; C07D 409/04; C07D 471/04

(52) U.S. Cl. CPC C07D 241/42; C07D 239/28; C07D 405/04; C07D 409/04; C07D 471/04

(57) ABSTRACT

A method for producing a quinoxaline includes reacting an o-phenylenediamine of formula (A) and a sulfoxonium ylide of formula (B) in an organic solvent in the presence of an N-halosuccinimide, in the absence of a metal catalyst, to form a quinoxaline of Formula I. R₁ is a substituted or unsubstituted, saturated or unsaturated, optionally heteroatom substituted alkyl group, R₂ is a benzene, a bromobenzene, a nitrobenzene, a furan, a cyclopropane, or a cyclohexane, and X is a carbon (C) or a nitrogen (N).

### BACKGROUND

### Technical Field

The present disclosure is directed to a method, more particularly, a method for producing a quinoxaline from o-phenylenediamines and sulfoxonium ylides at room temperature.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

The study of quinoxaline and its derivatives has become a subject of interest in recent years due to their wide variety of biological activities and therapeutic applications. Natural quinoxalines are considered rare such that most quinoxalines are synthetically produced. Various synthetic methods have been developed for the synthesis of quinoxalines. Among these methods, the reaction of ketones or alkynes with 1,2-diamines through an oxidation process, oxidative-cyclization of phenacyl bromides with 1,2-diamines, and oxidation of vicinal diols or α-hydroxy ketones with 1,2-diamines, have broadly been employed. Additional methods include a condensation reaction between a 1,2-diaminobenzene and a dicarbonyl compound, accompanied by an oxidation step to form the final quinoxaline ring structure and use of metal or enzyme catalysts. Enzyme catalysts, such as laccase and peroxidase, and metal catalysts, often a transition metal like copper, manganese, or palladium, facilitate the oxidation step necessary for cyclization to form the quinoxaline ring. Use of enzyme and metal catalysts, however, present several disadvantages including, but not limited to, potential toxicity of the metal catalyst, difficulty in catalyst separation and recovery from the reaction mixture, potential for metal contamination in the final product, high cost of enzyme and metal catalysts, difficult manufacturing due to the need for a careful control of reaction parameters, and the need for harsh reaction conditions which can be environmentally unfriendly. Thus, an efficient method of synthesis of quinoxalines that does not require the use of enzyme or metal catalysts is desired.

Similarly, the study of substituted pyrazines and its derivatives has increased recently due to their wide range of biological properties, including antibacterial, antiviral, and anticancer properties. Conventionally, synthesis of substituted pyrazines involves a metal-catalyzed cycloaddition where the azirine ring opens up and reacts with the triazole, leading to the formation of the pyrazine ring with substituents. Rhodium is frequently used as the metal catalyst due to its ability to facilitate the cyclization reaction between azirines and triazoles, leading to the formation of the pyrazine ring, however palladium and copper may also be used. As described above, the use of metal catalysts poses significant disadvantages in the synthesis of synthetic compounds. Moreover, pyrazine synthesis presents several other disadvantages, other than use of metal catalysts, such as low yields, complex multi-step reaction pathways, difficulty in controlling product mixtures, generation of significant byproducts, and limited availability of starting materials making. It, therefore, may be challenging to achieve efficient and environmentally friendly production of pyrazines, especially when aiming for specific substituted pyrazine derivatives. Thus, an efficient method of synthesis of substituted pyrazines that produces high yield and does not require the use of a metal catalyst is desired.

Accordingly, an objective of the present disclosure is to provide a green, environmentally friendly, and effective synthesis method with operational simplicity, mild reaction conditions, atom economy, and high yields for quinoxalines and tri-substituted pyrazines that may circumvent the stated disadvantages.

### SUMMARY

In an exemplary embodiment, a method for producing a quinoxaline is described. The method comprises reacting an o-phenylenediamine of Formula (A) and a sulfoxonium ylide of Formula (B)

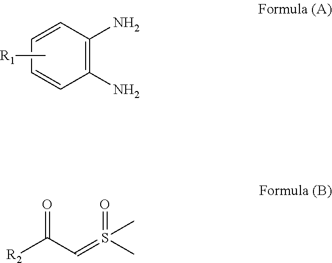

in an organic solvent in the presence of an N-halosuccinimide, in the absence of a metal catalyst, to form a quinoxaline of Formula I. R₁ is a substituted or unsubstituted, saturated or unsaturated, optionally heteroatom substituted alkyl group. R₂ is a benzene, a bromobenzene, a nitrobenzene, a furan, a cyclopropane, or a cyclohexane, and X is a carbon (C) or a nitrogen (N).

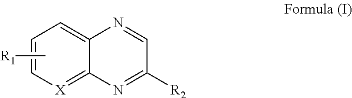

In some embodiments, the N-halosuccinimide is at least one selected from the group consisting of n-chlorosuccinimide (NCS), N-bromosuccinimide (NBS), and N-iodosuccinimide (NIS).

In some embodiments, the organic solvent is at least one selected from the group consisting of dichloromethane (DCM), acetone, chloroform, dimethylformamide (DMF), dimethyl sulfoxide (DMSO), tetrahydrofuran (THF), acetonitrile (ACN), and benzotrifluoride.

In some embodiments, the sulfoxonium ylide is a β-ketosulfoxonium ylide.

In some embodiments, the N-halogenated reagent is NIS.

In some embodiments, the organic solvent is DCM.

In some embodiments, the method of reacting the o-phenylenediamine comprises stirring the reaction mixture at a temperature of 20 to 25 degrees Celsius (° C.) for 1 to 3 hours (h).

In some embodiments, the method further comprises extracting an organic layer from a reaction mixture formed by reacting the o-phenylenediamine of Formula (A) and the sulfoxonium ylide of Formula (B) with ethyl acetate, and drying the organic layer with a drying agent to form a dried liquid mixture comprises the quinoxaline of Formula I. The drying agent is at least one selected from the group consisting of anhydrous sodium sulfate, anhydrous magnesium sulfate, calcium chloride, and calcium sulfate.

In some embodiments, the method of reacting the o-phenylenediamine of Formula (A) and the sulfoxonium ylide of Formula (B) is carried out with a sulfoxonium ylide to o-phenylenediamine molar ratio of 1:5 to 5:1.

In some embodiments, R₁ is a benzene, a pyridine, a xylene, or a methoxy benzene.

In another embodiment, a method of producing a tri-substituted pyrazine is described. The method comprises reacting a diaminomaleonitrile and a sulfoxonium ylide (B)

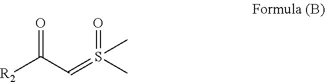

in an organic solvent in the presence of an N-halosuccinimide, in the absence of a metal catalyst, to form a tri-substituted pyrazine of Formula II. R₂ is a tert-butyl group, a benzene, a furan, or a methoxy benzene.

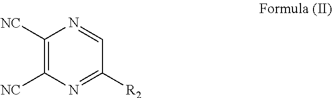

In some embodiments, the N-halosuccinimide is at least one selected from the group consisting of n-chlorosuccinimide (NCS), N-bromosuccinimide (NBS), and N-iodosuccinimide (NIS).

In some embodiments, the organic solvent is at least one of selected from the group consisting of dichloromethane (DCM), acetone, chloroform, dimethylformamide (DMF), dimethyl sulfoxide (DMSO), tetrahydrofuran (THF), acetonitrile (ACN), and benzotrifluoride.

In some embodiments, the sulfoxonium ylide is a β-ketosulfoxonium ylide.

In some embodiments, the N-halogenated reagent is NIS.

In some embodiments, the organic solvent is DCM.

In some embodiments, the method of reacting the diaminomaleonitrile and the sulfoxonium ylide comprises stirring the reaction mixture at a temperature of 20 to 25° C. for 1 to 3 h.

In some embodiments, the method of producing a tri-substituted pyrazine further comprises extracting an organic layer in the reaction mixture with ethyl acetate and drying the organic layer with a drying agent to obtain tri-substituted pyrazine of Formula II. The drying agent is at least one selected from the group consisting of anhydrous sodium sulfate, anhydrous magnesium sulfate, calcium chloride, and calcium sulfate.

In some embodiments, the method of reacting the diaminomaleonitrile and the sulfoxonium ylide is carried out with a sulfoxonium ylide to diaminomaleonitrile molar ratio of 1:5 to 5:1.

In some embodiments, R₂ in Formula (II) and Formula (B) is a methoxy benzene.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a method flowchart for synthesis of quinoxalines, according to certain embodiments.

FIG. 1B is a method flowchart for synthesis of tri-substituted pyrazine, according to certain embodiments.

FIG. 2 shows a synthesis scheme of quinoxalines, according to certain embodiments.

FIG. 3 shows a synthesis scheme of tri-substituted pyrazines. according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “compound” refers to a chemical entity, regardless of its phase—solid, liquid, or gaseous—as well as its state-crude mixture, purified, or isolated.

As used herein, the term “ultrasonication” or “sonication” refers the process of using sound waves, typically at ultrasonic frequencies, to agitate particles within a liquid sample, effectively mixing solutions, breaking apart clumps, or extracting compounds from materials like plant tissues

As used herein, the term “functional group” indicates specific groups of atoms within a molecular structure that are accountable for the characteristic chemical reactions and chemical properties of that structure. Suitable examples of functional groups include hydrocarbons, groups containing halogen, groups containing oxygen (e.g., alcohol, acid, ketone, aldehydes), groups containing nitrogen (e.g., nitrile, amines, amides), groups containing silicon (e.g., silanes) and groups containing phosphorus and sulfur.

As used herein, the term “attach” or “attachment” refers to linking or uniting by a bond, link, force, or tie in order to keep two or more parts together, which encompasses either direct or indirect attachment such that, for example, a first compound is directly bound to a second compound or material, and the embodiments wherein one or more intermediate compounds, and in particular molecules, are disposed between the first compound and the second compound or material.

As used herein, “alkyl group” or “alkyl” refers to monovalent saturated aliphatic hydrocarbyl groups having from 1 to 10 carbon atoms and such as 1 to 6 carbon atoms, or 1 to 5, or 1 to 4, or 1 to 3, or 1 to 2 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂) CH—), t-butyl (t-Bu) ((CH₃)₃C—), n-pentyl (CH₃CH₂CH₂CH₂CH₂—), and neopentyl ((CH₃)₃CCH₂—).

As used herein, the term “aromatic compounds” or “aromatic rings” refers to hydrocarbon rings that, by the theory of Hückel, have a cyclic, delocalized (4n+2) pi-electron system. Non-limiting examples of aromatic compounds include benzene, benzene derivatives, and compounds having at least one benzene ring in their chemical structure.

As used herein, the term “atom economy” or “atom efficiency” refers to a measure of how efficiently a chemical reaction converts the atoms in its reactants into the desired product, essentially indicating the percentage of atoms from the starting materials that end up in the final useful product, with a higher atom economy signifying a more efficient and environmentally friendly reaction process. Atom economy is calculated by dividing the mass of the desired product by the total mass of all reactants, expressed as a percentage. A “good” atom economy is depicted by a high percentage, with the ideal being a 100% atom economy. Atom economy measures the environmental friendliness of a chemical process because it relates to the minimization of waste by ensuring most of the reactant atoms are utilized and not lost as byproducts, thus aligning with the principles of sustainability and environmental responsibility

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic, or it may have altered reactivity as compared to the parent compound. Derivatization (i.e., modification) may involve the substitution of one or more moieties within the molecule (e.g., a change in a functional group). The term “derivative” also includes conjugates, and prodrugs of a parent compound (i.e., chemically modified derivatives that can be converted into the original compound under physiological conditions).

As used herein, “thermal stability” refers to how well a compound can withstand high temperatures without decomposing. Compounds with low thermal stability may react unpredictably or decompose when heated, which can lead to undesirable outcomes.

As used herein, “yield rate” or “percent yield,” refers to the ratio of the actual amount of product obtained from a chemical reaction (actual yield) compared to the maximum theoretical amount of product that could be produced (theoretical yield), expressed as a percentage.

A weight percent (wt. %) of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100 wt. %.

The present disclosure is intended to include all hydration states of a given compound or formula, unless otherwise noted or when heating a material.

In addition, the present disclosure is intended to include all isotopes of atoms occurring in the present compounds and complexes. Isotopes include those atoms having the same atomic number but different mass numbers. By way of general example, and without limitation, isotopes of hydrogen include deuterium and tritium, and isotopes of carbon include ¹³C and ¹⁴C. Isotopes of oxygen include ¹⁶O, ¹⁷O, and ¹⁸O. Isotopically labeled compounds of the disclosure may generally be prepared by conventional techniques known to those skilled in the art or by processes analogous to those described herein, using an appropriate isotopically labeled reagent in place of the non-labeled reagent otherwise employed.

Aspects of the present disclosure are directed to a green methodology for N-iodosuccinimide (NIS) mediated synthesis of quinoxalines at room temperature without the use of a metal catalyst. The method of the present disclosure for synthesizing quinoxaline derivatives at room temperature is green, environmentally friendly, and effective.

In one aspect, a method for producing a quinoxaline is described. Quinoxaline and derivatives thereof are an important class of heterocycle compounds, where N replaces some carbon atoms in the ring of naphthalene. Its molecular formula is C₈H₆N₂, formed by the fusion of two aromatic rings, benzene and pyrazine. The aromatic structure of quinoxaline allows for a wide range of potential biological activities due to its electron delocalization across the fused benzene and pyrazine rings, making it a valuable scaffold in medicinal chemistry for developing drugs with antibacterial, anticancer, antiviral, and anti-inflammatory properties, among others. The aromatic nature of quinoxaline further enables diverse substitutions and interactions with biological targets, enhancing its therapeutic potential. Quinoxaline-based chemicals may also be employed in organic electronics, because quinoxaline can be used as an electron-acceptor unit (A) which, being combined with different electron-donor blocks (D), make up for donor-acceptor (D-A) type systems. Such D-A quinoxaline-based compounds are promising materials for different photovoltaics and optoelectronics applications like dye-sensitized solar cells (DSSCs), bulk heterojunction solar cells, organic thin film transistors, fluorescence chemosensors, fluorescence imaging, organic light emitting diodes (OLEDs), and electrochromic devices. Quinoxaline is also an important intermediate in synthetic chemistry, as its reactivity enables the formation of more complicated compounds and slight modifications to quinoxaline's structure allows for the formation of different moieties with unique characteristics.

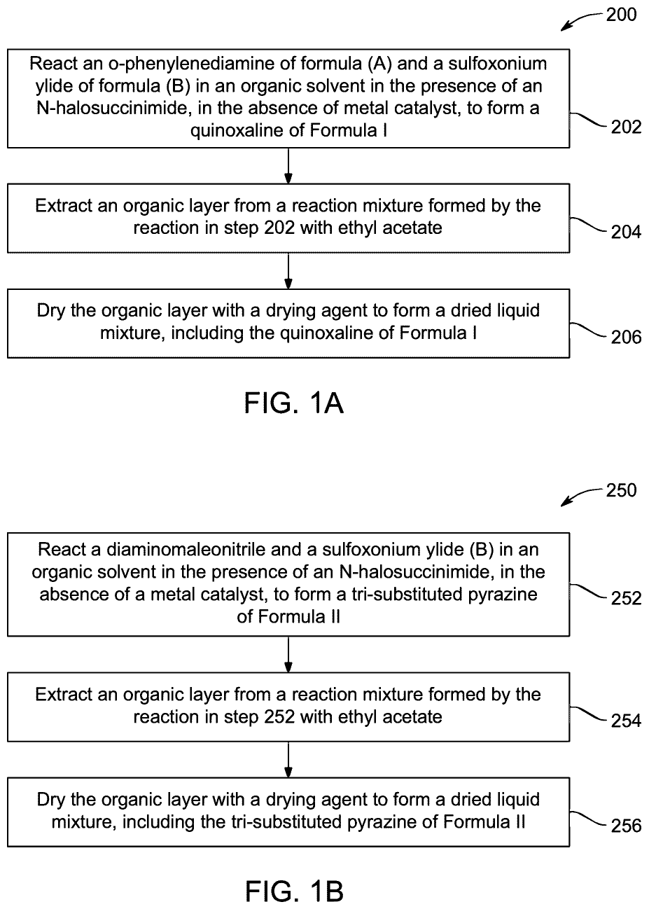

As presented in FIG. 1A, a schematic flow diagram of the method 200 for producing a quinoxaline is illustrated. The order in which the method 200 is described is not intended to be construed as a limitation, and any number of the described method steps may be combined in any order to implement the method 200. Additionally, individual steps may be removed or skipped from the method 200 without departing from the spirit and scope of the present disclosure.

At step 202, the method 200 comprises reacting an o-phenylenediamine of formula (A) and a sulfoxonium ylide of formula (B) in an organic solvent in the presence of an N-halosuccinimide, in the absence of a metal catalyst to form a quinoxaline of Formula (I).

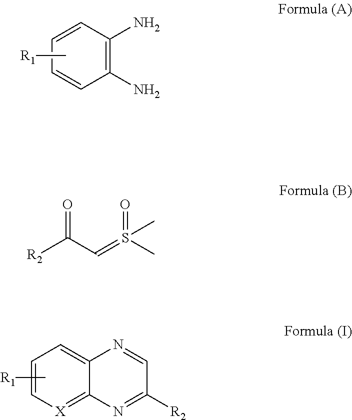

A sulfoxonium ylide is a reactive chemical intermediate composed of a sulfur atom bound to an oxygen atom and a negatively charged carbon. It is commonly generated by deprotonating a sulfoxide and is employed in chemical synthesis for reactions such as nucleophilic substitution and alkene production. Conventionally, diazo compounds are used the synthesis of quinoxalines because the diazo compounds act as a nucleophile by attacking electrophilic centers due to their electron-rich nature. Use of a diazo compound, however, generally involves a metal catalyst to control the reactivity of the obtained intermediates. Additionally, the use of diazo compounds on a large scale is challenging due to rapid heat liberation during the reaction and the generation of nitrogen gas as a byproduct. Diazo compounds are also highly reactive, increasing the risk of volatile reaction conditions. In contrast, sulfoxonium ylides are a better and more practical alternative to diazo compounds in the synthesis of quinoxalines. Sulfoxonium ylides offer superior thermal stability over diazo compounds, meaning they can withstand relatively high temperatures without decomposing, making them easier to handle due to their decreased risk of volatile reactions as compared with diazo compounds. Suitable examples of sulfoxonium ylides include, but are not limited to, methylsulfoxonium ylide, phenylsulfoxonium ylide, ethylsulfoxonium ylide, benzylsulfoxonium ylide, β-ketosulfoxonium ylide, and tert-butylsulfoxonium ylide. In a preferred embodiment, the sulfoxonium ylide is a β-ketosulfoxonium ylide.

In some embodiments, the N-halosuccinimide is at least one selected from N-chlorosuccinimide (NCS), N-bromosuccinimide (NBS), and NIS. N-halosuccinimides are a group of organic compounds that are used in a variety of organic synthesis reactions such as chlorination, oxidation, and halogenation. N-halosuccinimides can also mediate or catalyze chemical reactions including, but not limited to, halocyclizations, formation of heterocyclic systems, and rearrangements. N-halosuccinimides can also be used to protect various functional groups. N-halosuccinimide are inexpensive, easy to handle, relatively stable, and metal-free compounds, offering a beneficial alternative to the use of metal or enzymatic catalysts in the synthesis of quinoxalines.

An organic solvent is a carbon-based substance employed to dissolve other substance(s). In some embodiments, the organic solvent is at least one selected from the group including dichloromethane (DCM), acetone, chloroform, dimethylformamide (DMF), dimethyl sulfoxide (DMSO), tetrahydrofuran (THF), acetonitrile (ACN), and benzotrifluoride. In alternate embodiments, organic solvents may be selected from, but not limited to, methanol, ethanol, dimethylacetamide, isopropanol (IPA), benzene, hexane, carbon tetrachloride, toluene, diethyl ether, and mixtures thereof. In a preferred embodiment, the organic solvent is DCM.

In some embodiments, the method of reacting the o-phenylenediamine of formula (A) and the sulfoxonium ylide of formula (B) is carried out with a sulfoxonium ylide to o-phenylenediamine molar ratio of 1:5 to 5:1, preferably 1:4 to 4:1, preferably 1:3 to 3:1, preferably 1:2 to 2:1, and most preferably 1:1. In some embodiments, the method of reacting the o-phenylenediamine of formula (A) and the sulfoxonium ylide of formula (B) comprises stirring the reaction mixture at a temperature of 20 to 25° C. for 1 to 3 h. In another embodiment, the method of reacting the o-phenylenediamine of formula (A) and the sulfoxonium ylide of formula (B) comprises stirring the reaction mixture at a temperature of 20.5 to 24.5° C., preferably 21 to 24° C., preferably 21.5 to 23.5° C., most preferably 22 to 23° C. In yet another embodiment, the method of reacting the o-phenylenediamine of formula (A) and the sulfoxonium ylide of formula (B) comprises stirring the reaction mixture for 1.25 to 2.75 h, preferably 1.5 to 2.5 h, preferably 1.75 to 2.25 h, most preferably 2 h. In a preferred embodiment, the reaction mixture is stirred at a temperature of 22 to 23° C. for 2 h. In some embodiments, the reaction mixture is stirred by any known methods such as manual swirling, magnetic stirring, vortexing, sonicating, or a combination thereof. In some embodiments, the reaction mixture is stirred at a rate of 200 to 1000 rotations per minute (rpm), preferably 250 to 950 rpm, preferably 300 to 900 rpm, preferably 350 to 850 rpm, preferably 400 to 800 rpm, preferably 450 to 750 rpm, most preferably 500 to 600 rpm. In some embodiments, the reaction mixture is stirred via sonication for 1 to 3 h at a frequency of 15 to 50 kHz. In other embodiments, the reaction mixture is sonicated for 1.25 to 2.75 h, preferably 1.5 to 2.5 h, preferably 1.75 to 2.25 h, most preferably 2 h. In other embodiments, the reaction mixture is sonicated for 1 to 3 h at a frequency of 20 to 45 kHz, preferably 20 to 40 kHz, preferably 20 to 35 kHz, preferably 20 to 30 kHz, preferably 20 to 25 kHz, most preferably 20 kHz.

In some embodiments, R₁ is a substituted or unsubstituted, saturated or unsaturated, optionally heteroatom substituted alkyl group, R₂ is a benzene, bromobenzene, a nitrobenzene, a furan, a cyclopropane, or cyclohexane, and X is a carbon (C) or nitrogen (N). In some embodiments, R₁ is a benzene, a pyridine, a xylene, or a methoxy benzene.

At step 204, the method 200 comprises extracting an organic layer from a reaction mixture formed with ethyl acetate. In other embodiments, extraction solvents like acetone, tetrahydrofuran (THF), dimethylsulfoxide (DMSO), MeOH, EtOH, and acetonitrile (ACN), alone or in combination, may also be used.

At step 206, the method 200 comprises drying the organic layer with a drying agent to form a dried liquid mixture comprising the quinoxaline of Formula I. The drying agent is selected from anhydrous sodium sulfate, anhydrous magnesium sulfate, calcium chloride, and calcium sulfate. In a preferred embodiment, the drying agent is anhydrous sodium sulfate. When added to the mixture, sodium sulfate binds to water via ion-dipole interactions, producing hydrated sodium sulfate. This eliminates moisture from the organic solvent, and after enough drying, the sodium sulfate is filtered, leaving only a dry organic phase. Sodium sulfate may be separated using any known separation techniques such as decantation, centrifugation, gravity filtration, vacuum filtration, pressure filtration, membrane filtration, gas flotation, capacitance-based separation, or microfiltration. Anhydrous sodium sulfate is a preferred drying agent because it is easy to separate from the organic layer because it is easily identifiable when it becomes saturated due to clumping, as compared to other drying agents like anhydrous magnesium sulfate, which may take a longer time to fully dry the organic layer.

In some embodiments, the method for producing a quinoxaline achieves a yield rate of at least 60%. In another embodiment, the method for producing a quinoxaline achieves a yield rate of at least 62%, preferably at least 64%, preferably at least 66%, preferably at least 68%, preferably at least 70%, preferably at least 72%, preferably at least 74%, preferably at least 76%, preferably at least 78%, preferably at least 80%, preferably at least 82%, preferably at least 84%, preferably at least 86%, preferably at least 88%, preferably at least 90%, most preferably at least 91%.

In some embodiments, the method for producing a quinoxaline achieves an atom economy of at least 50%. When synthesizing quinoxaline using a sulfoxonium ylide, the primary byproduct is typically the corresponding sulfoxide formed by the loss of the ylide's carbanion during the cyclization step. In the present method, the sulfoxonium ylide generates non-volatile dimethyl sulfoxide (DMSO) as a byproduct, which increases the safety of the quinoxaline synthesis and can be easily separated after the reaction allowing for increased efficiency. In another embodiment, the method for producing a quinoxaline achieves a yield rate of at least 52%, preferably at least 54%, preferably at least 56%, preferably at least 58%, preferably at least 60%, preferably at least 62%, preferably at least 64%, preferably at least 66%, preferably at least 68%, preferably at least 70%, preferably at least 72% preferably at least 74%, preferably at least 76%, preferably at least 78%, most preferably at least 80%.

In a second aspect of the present disclosure, a method for producing a tri-substituted pyrazine is described. Referring to FIG. 1B, a schematic flow diagram of the method 250 for producing a tri-substituted pyrazine is illustrated. The order in which the method 250 is described is not intended to be construed as a limitation, and any number of the described method steps may be combined in any order to implement the method 250. Additionally, individual steps may be removed or skipped from the method 250 without departing from the spirit and scope of the present disclosure.

At step 202, the method 250 comprises reacting a diaminomaleonitrile and a sulfoxonium ylide (B) in an organic solvent in the presence of an N-halosuccinimide, in the absence of a metal catalyst, to form a tri-substituted pyrazine of Formula II.

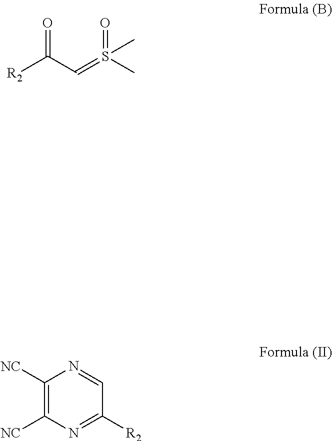

In some embodiments, R₂ is a tert-butyl group, a benzene, a furan, or a methoxy benzene. In a preferred embodiment, R₂ in Formula (II) and Formula (B) is a methoxy benzene.

In substituted pyrazine synthesis, diazo compounds are primarily used as a starting material to generate reactive carbenoid intermediates through a [3+2] cycloaddition reaction, often leading to the formation of pyrazine rings by subsequent ring closure or rearrangement steps. Use of a diazo compound, however, generally involves a metal catalyst to control the reactivity of the obtained intermediates. Additionally, the use of diazo compounds on a large scale is challenging due to rapid heat liberation during the reaction and the generation of nitrogen gas as a byproduct. Diazo compounds are also highly reactive, increasing the risk of volatile reaction conditions.

In contrast, diaminomaleonitrile (DAMN) is an organic compound composed of two amino groups and two nitrile groups bonded to a central alkene unit. DAMN provides a versatile and inexpensive building block for synthesizing a variety of organic compounds, including but not limited to, substituted pyrazines, quinoxalines, and the like.

Further, sulfoxonium ylides are a better and more practical alternative to diazo compounds in the synthesis of substituted pyrazines. Sulfoxonium ylides offer superior thermal stability over diazo compounds, meaning they can withstand relatively high temperatures without decomposing, making them easier to handle due to their decreased risk of volatile reactions as compared with diazo compounds. Suitable examples of sulfoxonium ylides include, but are not limited to, methylsulfoxonium ylide, phenylsulfoxonium ylide, ethylsulfoxonium ylide, benzylsulfoxonium ylide, β-ketosulfoxonium ylide, and tert-butylsulfoxonium ylide. In a preferred embodiment, the sulfoxonium ylide is a β-ketosulfoxonium ylide.

In some embodiments, the N-halosuccinimide is at least one selected from the group consisting of NCS, NBS, and NIS. N-halosuccinimides are a group of organic compounds that are used in a variety of organic synthesis reactions such as chlorination, oxidation, and halogenation. N-halosuccinimides can also mediate or catalyze chemical reactions including, but not limited to, halocyclizations, formation of heterocyclic systems, and rearrangements. N-halosuccinimides can also be used to protect various functional groups. N-halosuccinimide are inexpensive, easy to handle, relatively stable, and metal-free compounds, offering a beneficial alternative to the use of metal catalysts in the synthesis of tri-substituted pyrazines. In a preferred embodiment, the N-halosuccinimide is NIS.

In some embodiments, the organic solvent is at least one of selected from the group consisting of DCM, acetone, chloroform, DMF, DMSO, THF, ACN, and benzotrifluoride. In alternate embodiments, organic solvents may be selected from, but not limited to, methanol, ethanol, dimethylacetamide, IPA, benzene, hexane, carbon tetrachloride, toluene, diethyl ether, and mixtures thereof. In a preferred embodiment, the organic solvent is DCM.

In some embodiments, the method of reacting diaminomaleonitrile and sulfoxonium ylide (B) is carried out with a sulfoxonium ylide to diaminomaleonitrile molar ratio of 1:5 to 5:1, preferably 1:4 to 4:1, preferably 1:3 to 3:1, preferably 1:2 to 2:1, and most preferably 1:1. In some embodiments, the reacting comprises stirring the reaction mixture at a temperature of 20 to 25° C. for 1 to 3 h. In another embodiment, the reacting comprising stirring the reaction mixture at a temperature of 21 to 24° C., most preferably 22 to 23° C. In another embodiment, the reacting comprising stirring the reaction mixture for 1.25 to 2.75 h, preferably 1.5 to 2.5 h, preferably 1.75 to 2.25 h, most preferably 2 h. In a preferred embodiment, the reacting comprises stirring the reaction mixture at a temperature of 22 to 23° C. for 2 h. In some embodiments, the reaction mixture is stirred by any known methods such as manual swirling, magnetic stirring, vortexing, sonicating, or a combination thereof. In some embodiments, the reaction mixture is stirred at a rate of 200 to 1000 rpm, preferably 250 to 950 rpm, preferably 300 to 900 rpm, preferably 350 to 850 rpm, preferably 400 to 800 rpm, preferably 450 to 750 rpm, most preferably 500 to 600 rpm. In some embodiments, the reaction mixture is stirred via sonication for 1 to 3 h at a frequency of 15 to 50 kHz. In other embodiments, the reaction mixture is sonicated for 1.25 to 2.75 h, preferably 1.5 to 2.5 h, preferably 1.75 to 2.25 h, most preferably 2 h. In other embodiments, the reaction mixture is sonicated for 1 to 3 h at a frequency of 20 to 45 kHz, preferably 20 to 40 kHz, preferably 20 to 35 kHz, preferably 20 to 30 kHz, preferably 20 to 25 kHz, most preferably 20 kHz.

At step 204, the method 250 comprises extracting an organic layer in the reaction mixture with ethyl acetate. Optionally, other extraction solvents like acetone, tetrahydrofuran (THF), DMSO, MeOH, EtOH, and ACN, alone or in combination, as obvious to a person skilled in the art, may also be used.

At step 206, the method 250 comprises drying the organic layer with a drying agent to obtain tri-substituted pyrazine of Formula II. The drying agent is at least one selected from the group including anhydrous sodium sulfate, anhydrous magnesium sulfate, calcium chloride, and calcium sulfate. In a preferred embodiment, the drying agent is anhydrous sodium sulfate. During drying, anhydrous sodium sulfate absorbs water molecules from the organic layer. When added to the mixture, sodium sulfate binds to water via ion-dipole interactions, producing hydrated sodium sulfate. This eliminates moisture from the organic solvent, and after enough drying, the sodium sulfate is filtered, leaving only a dry organic phase. Sodium sulfate may be separated using any known separation techniques such as decantation, centrifugation, gravity filtration, vacuum filtration, pressure filtration, membrane filtration, gas flotation, capacitance-based separation, or microfiltration.

In some embodiments, the method for producing a tri-substituted pyrazine achieves a yield rate of at least 60%. In another embodiment, the method for producing a quinoxaline achieves a yield rate of at least 62%, preferably at least 64%, preferably at least 66%, preferably at least 68%, preferably at least 70%, preferably at least 72%, preferably at least 74%, preferably at least 76%, preferably at least 78%, preferably at least 80%, preferably at least 82%, preferably at least 84%, preferably at least 86%, preferably at least 88%, most preferably at least 89%

In some embodiments, the method for producing a tri-substituted pyrazine achieves an atom economy of at least 60%. When synthesizing tri-substituted pyrazine using a sulfoxonium ylide, the primary byproduct is typically the corresponding sulfoxide formed by the loss of the ylide's carbanion during the cyclization step. In the present method, the sulfoxonium ylide generates non-volatile dimethyl sulfoxide (DMSO) as a byproduct, which increases the safety of the quinoxaline synthesis and can be easily separated after the reaction allowing for increased efficiency. In another embodiment, the method for producing a quinoxaline achieves a yield rate of at least 62%, preferably at least 64%, preferably at least 66%, preferably at least 68%, preferably at least 70%, preferably at least 72%, preferably at least 74%, preferably at least 76%, preferably at least 78%, preferably at least 80%, preferably at least 82% preferably at least 84%, most preferably at least 86%.

### EXAMPLES

The following examples demonstrate a method for producing quinoxaline from o-phenylenediamines and sulfoxonium ylides at room temperature. The examples are provided solely for illustration and are not to be construed as limitations of the present disclosure, as many variations thereof are possible without departing from the spirit and scope of the present disclosure.

### Example 1: Procedure for the Synthesis of Quinoxalines

A total of 23 molecules were synthesized using the current methodology. A mixture of β-ketosulfoxonium ylides (1.0 mmol), o-phenylenediamines (1.0 mmol), and N-iodosuccinimide (NIS) (1.8 mmol) in dichloromethane (DCM) (CH₂Cl₂) (5 mL) was stirred at room temperature for 2 h. After thin layer chromatography (TLC) monitoring, the solvent was removed under pressure. Water (10 mL) was added to the reaction mixture and extracted with ethyl acetate (3×10 mL). The combined organic layer was dried over anhydrous Na₂SO₄, and the solvent was concentrated under vacuum. The residue was purified by silica gel column chromatography (5 to 10% EtOAc in hexane) to afford the corresponding pure products.

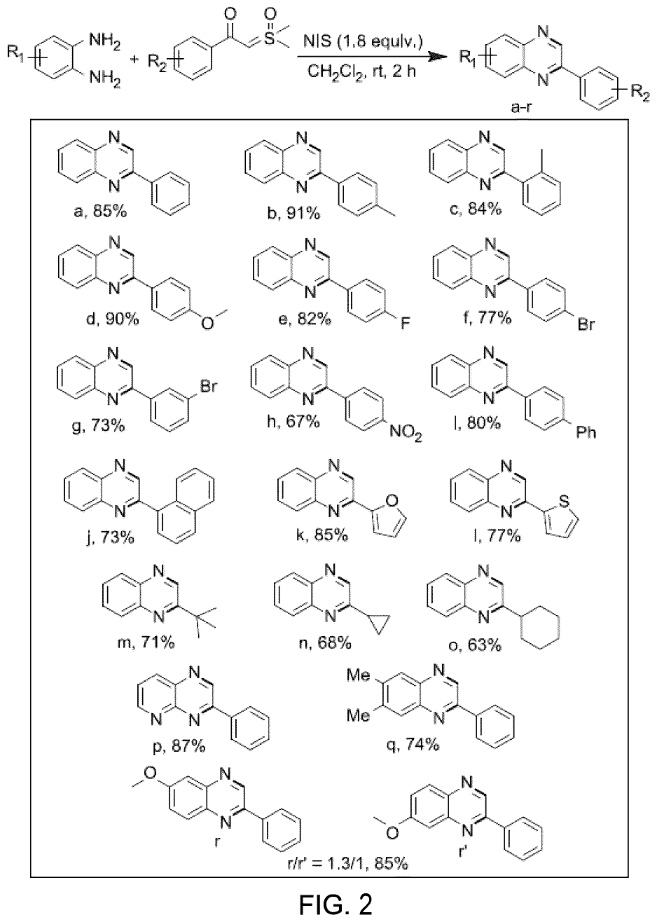

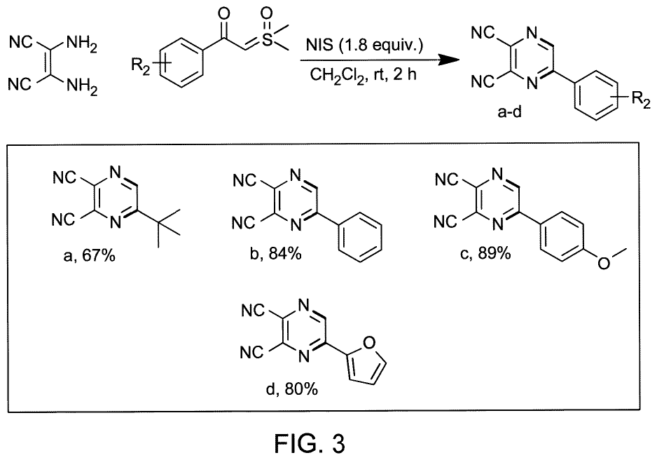

A mixture of β-ketosulfoxonium ylides (1.0 mmol), o-phenylenediamines (1.0 mmol), and N-iodosuccinimide (NIS, 1.8 mmol) in dichloromethane (DCM) (CH₂Cl₂, 5 milliliters (mL)) was stirred at room temperature for 2 h. The reaction progress was monitored by TLC. Upon completion, the solvent was removed under reduced pressure. Water (10 mL) was added to the reaction mixture, which was then extracted with ethyl acetate (EtOAc) (3×10 mL). The combined organic layers were dried over anhydrous sodium sulfate (Na₂SO₄), and the solvent was evaporated under vacuum. The residue was purified by silica gel column chromatography (5 to 10% ethyl acetate (EtOAc) in hexane) to afford the desired products with increased purity. FIG. 2 shows the synthesis scheme of quinoxalines (3a-3r′), and FIG. 3 shows the synthesis scheme of tri-substituted pyrazines (5a-5d).

Numerous modifications and variations of the present disclosure are possible in light of the above teachings. It is, therefore, to be understood that within the scope of the appended claims, the invention may be practiced otherwise than as specifically described herein.

1. A method for producing a quinoxaline, comprising:
reacting an o-phenylenediamine of formula (A) and a sulfoxonium ylide of formula (B) 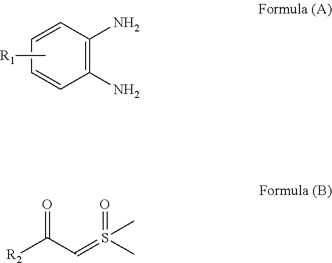
in an organic solvent in the presence of an N-halosuccinimide, in the absence of a metal catalyst, to form a quinoxaline of Formula I 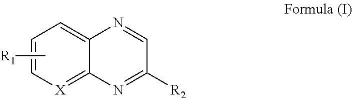
wherein
R₁ is a substituted or unsubstituted, saturated or unsaturated, optionally heteroatom substituted alkyl group,
R₂ is a benzene, a bromobenzene, a nitrobenzene, a furan, a cyclopropane, or a cyclohexane, and
X is a carbon (C) or a nitrogen (N).

2. The method of claim 1, wherein the N-halosuccinimide is at least one selected from the group consisting of n-chlorosuccinimide (NCS), N-bromosuccinimide (NBS), and N-iodosuccinimide (NIS).

3. The method of claim 1, wherein the organic solvent is at least one selected from the group consisting of dichloromethane (DCM), acetone, chloroform, dimethylformamide (DMF), dimethyl sulfoxide (DMSO), tetrahydrofuran (THF), acetonitrile (ACN), and benzotrifluoride.

4. The method of claim 1, wherein the sulfoxonium ylide is a β-ketosulfoxonium ylide.

5. The method of claim 2, wherein the N-halogenated reagent is NIS.

6. The method of claim 1, wherein the organic solvent is DCM.

7. The method of claim 1, wherein the reacting comprises stirring the reaction mixture at a temperature of 20 to 25° C. for 1 to 3 h.

8. The method of claim 1, further comprising:
extracting an organic layer from a reaction mixture formed by the reacting with ethyl acetate; and
drying the organic layer with a drying agent to form a dried liquid mixture comprising the quinoxaline of Formula I,
wherein the drying agent is at least one selected from the group consisting of anhydrous sodium sulfate, anhydrous magnesium sulfate, calcium chloride, and calcium sulfate.

9. The method of claim 1, wherein the reacting is carried out with a sulfoxonium ylide to o-phenylenediamine molar ratio of 1:5 to 5:1.

10. The method of claim 1, wherein R₁ is a benzene, a pyridine, a xylene, or a methoxy benzene.

11. A method for producing a tri-substituted pyrazine, comprising:
reacting a diaminomaleonitrile and a sulfoxonium ylide (B) 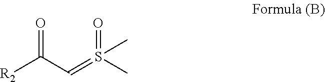
in an organic solvent in the presence of an N-halosuccinimide, in the absence of a metal catalyst, to form a tri-substituted pyrazine of Formula II 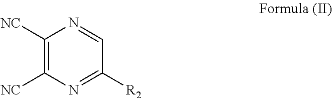
wherein
R₂ is a tert-butyl group, a benzene, a furan, or a methoxy benzene.

12. The method of claim 11, wherein the N-halosuccinimide is at least one selected from the group consisting of NCS, NBS, and NIS.

13. The method of claim 11, wherein the organic solvent is at least one of selected from the group consisting of DCM, acetone, chloroform, DMF, DMSO, THE, ACN, and benzotrifluoride.

14. The method of claim 11, wherein the sulfoxonium ylide is a β-ketosulfoxonium ylide.

15. The method of claim 11, wherein the N-halogenated reagent is NIS.

16. The method of claim 11, wherein the organic solvent is DCM.

17. The method of claim 11, wherein the reacting comprises stirring the reaction mixture at a temperature of 20 to 25° C. for 1 to 3 h.

18. The method of claim 11, further comprising:
extracting an organic layer in the reaction mixture with ethyl acetate; and
drying the organic layer with a drying agent to obtain the tri-substituted pyrazine of Formula II,
wherein the drying agent is at least one selected from the group consisting of anhydrous sodium sulfate, anhydrous magnesium sulfate, calcium chloride, and calcium sulfate.

19. The method of claim 11, wherein the reacting is carried out with a sulfoxonium ylide to diaminomaleonitrile molar ratio of 1:5 to 5:1.

20. The method of claim 11, wherein R₂ is a methoxy benzene.
